(19) United States
(12) Patent Application Publication
(10) Pub. No.: US 2024/0139262 A1
(43) Pub. Date: May 2, 2024

(54) COMPLEX PROBIOTIC COMPOSITION AND METHOD FOR IMPROVING EXERCISE PERFORMANCE OF SUBJECT WITH LOW INTRINSIC AEROBIC EXERCISE CAPACITY

(71) Applicant: GRAPE KING BIO LTD, Taoyuan City (TW)

(72) Inventors: Chin-Chu CHEN, Taoyuan City (TW); Yen-Lien CHEN, Taoyuan City (TW); Shih-Wei LIN, Taoyuan City (TW); Yen-Po CHEN, Taoyuan City (TW); Ci-Sian WANG, Taoyuan City (TW); Yu-Hsin HOU, Taoyuan City (TW); Yang-Tzu SHIH, Taoyuan City (TW); Ching-Wen LIN, Taoyuan City (TW); Ya-Jyun CHEN, Taoyuan City (TW); Jia-Lin JIANG, Taoyuan City (TW); You-Shan TSAI, Taoyuan City (TW); Zi-He WU, Taoyuan City (TW)

(21) Appl. No.: 18/486,674

(22) Filed: Oct. 13, 2023

(30) Foreign Application Priority Data
Oct. 27, 2022 (TW) 111140883

(51) Int. Cl. A61K 35/747; A61K 35/745; A61P 3/04; A61P 3/08; A61P 43/00

(52) U.S. Cl. CPC A61K 35/747; A61K 35/745; A61P 3/04; A61P 3/08; A61P 43/00; C12R 2001/225

(57) ABSTRACT

The present disclosure relates to a complex probiotic composition and a method for improving exercise performance of a subject with low intrinsic aerobic exercise capacity. The complex probiotic composition, which includes Lactobacillus rhamnosus GKLC1, Bifidobacterium lactis GKK24 and Clostridium butyricum GKB7, administered to the subject with the low intrinsic aerobic exercise capacity in a continuation period, can effectively reduce serum lactic acid and serum urea nitrogen after aerobic exercise, reduce proportion of offal fat and/or increase liver and muscle glycogen contents, thereby being as an effective ingredient for preparation of various compositions.

### RELATED APPLICATION

This application claims priority to Taiwan Application Serial Number 111140883, filed Oct. 27, 2022, which is herein incorporated by reference.

### REFERENCE TO A SEQUENCE LISTING

A sequence listing is being submitted herein as xml format with the name “TSAI_SP5782_SEQ_LIST”, created on Oct. 3, 2023, with a file size of 5,040 bytes, the contents of which are herein incorporated by reference.

### BACKGROUND

### Field of Invention

The present disclosure relates to a probiotic composition and a method of using the same, in particular to a complex probiotic composition and a method for improving exercise performance of a subject with low intrinsic aerobic exercise capacity, thereby reducing serum lactic acid and serum urea nitrogen after aerobic exercise, reducing proportion of offal fat and/or increasing liver and muscle glycogen contents.

### Description of Related Art

Factors such as cardiorespiratory fitness, muscular fitness, flexibility, age, gender, heredity, body shape, drugs, environment, etc., all affect individual exercise performance. Although “acquired” efforts, such as training, diet control, etc., can improve overall exercise performance, studies have found that under the same conditions (e.g., gender, age, training content, etc.), exercise performance still be affected by “innate (or intrinsic) constitution”.

In the same-age and same-gender experimental mice from the same mother, it was found that exercise performance of the same batch of mice was not normal distribution, but linear distribution; that is, some mice were born with high exercise capacity, and some mice were born with low exercise capacity, and a gap between the most athletic and the least athletic was very large. If physique of those with low intrinsic aerobic exercise capacity can be improved or adjusted, a difference between them through acquired exercise training and those with high intrinsic aerobic exercise capacity can be narrowed.

Studies have found that, those with low intrinsic aerobic exercise capacity not only had poorer exercise performance than the same ethnic group, but also had poorer physiological composition and metabolic-related values, such as a high proportion of offal fat (e.g., epididymal fat pad), a high level of blood sugar, a low ratio of red muscle to white muscle, etc. For example, mice were selectively bred for high or low willingness to run on a voluntary wheel, it was found that the two animal groups with different innate exercise capacities had differences on health, aging and extended life span, as well as risk factors for chronic diseases including sensitivities to metabolic syndrome and cardiovascular complications.

A group with low intrinsic exercise capacity had significantly higher sensitivities to physiology, psychology, behavior and metabolic diseases than those with high intrinsic exercise capacity (Thyfault and Morris, 2017; The Journal of Physiology 595(14): 4909-4926). Therefore, if innate constitution may be improved or adjusted, in addition to improving exercise performance, it is also expected to improve the physiological state related to metabolism and effectively prevent the occurrence of diseases.

Probiotics are generally defined as bacteria that are beneficial to human health and can multiply in the human intestinal tract and are not pathogenic. Usually, the probiotics can regulate physique and change some metabolic pathways of the human body and achieve functions such as improving the overall gastrointestinal tract and metabolic capacity.

In recent years, many studies have suggested that probiotics have potential to enhance exercise performance. Studies have found that Lactiplantibacillus plantarum can beneficially improve exercise endurance performance of general mice and reduce accumulation of lactic acid in serum after exercise. Studies have also found that Lactobacillus salivarius can reduce urea nitrogen and creatine kinase in serum and increase glycogen in liver and muscle of general mice after exercise, and shorten a physiological recovery time after exercise.

However, current studies are only aimed at probiotics to improve exercise performance evaluation of the general population (i.e., the population with general aerobic exercise capacity), without taking into account exercise performance affected by “innate factors.” It is urgent to develop an improved complex probiotic composition and a method for improving exercise performance of a subject with low intrinsic aerobic exercise capacity.

### SUMMARY

Therefore, one aspect of the present disclosure provides a complex probiotic composition for improving exercise performance of a subject with low intrinsic aerobic exercise capacity, which includes Lactobacillus rhamnosus GKLC1, Bifidobacterium lactis GKK24 and Clostridium butyricum GKB7.

Moreover, another aspect of the present disclosure provides a method of improving exercise performance of a subject with low intrinsic aerobic exercise capacity, which includes administering an oral composition containing an effective dose of the complex probiotics to a subject in a continuation period, and the complex probiotics includes the aforementioned bacterial strains as an effective ingredient.

According to the above aspect of the present disclosure, a complex probiotic composition for improving exercise performance of a subject with low intrinsic aerobic exercise capacity is provided. In one embodiment, the complex probiotic composition for improving the exercise performance of the subject with the low intrinsic aerobic exercise capacity includes Lactobacillus rhamnosus GKLC1, Bifidobacterium lactis GKK24 and Clostridium butyricum GKB7. The Lactobacillus rhamnosus GKLC1 is deposited in the China General Microbiological Culture Collection Center (CGMCC, No. 3, Yard 1, West Beichen Road, Chaoyang District, Beijing, China) on Jan. 12, 2018 and assigned accession number CGMCC 15202, and the Bifidobacterium lactis GKK24 is deposited in the International Patent Organism Depositary, National Institute of Technology and Evaluation (NITE-IPOD, #122, 2-5-8 Kazusakamatari, Kisarazu-shi, Chiba, Japan) on Oct. 14, 2022 and assigned accession number NITE BP-03766, and the Clostridium butyricum GKB7 is deposited in the NITE-IPOD on May 22, 2023 and assigned accession number NITE BP-03889.

In the foregoing embodiments, the complex probiotic composition may be an oral composition, such as a pharmaceutical composition or a food composition. In the foregoing embodiments, the complex probiotic composition further includes a pharmaceutical and food-acceptable carrier, excipient and/or additive.

In the foregoing embodiments, a dosage form of the complex probiotic composition may be powder, lozenge, granule, suppository, microcapsule, ampoule, liquid spray, or suppository.

In the foregoing embodiments, improving the exercise performance of the subject with the low intrinsic aerobic exercise capacity includes reducing serum lactic acid and serum urea nitrogen after aerobic exercise, reducing proportion of offal fat and/or increasing liver and muscle glycogen contents.

According to another aspect of the present disclosure, a method of improving exercise performance of a subject with low intrinsic aerobic exercise capacity includes administering an oral composition containing an effective dose of the complex probiotics to the subject for at least 28 consecutive days, and the complex probiotics include Lactobacillus rhamnosus GKLC1 (CGMCC 15202), Bifidobacterium lactis GKK24 (NITE BP-03766) and Clostridium butyricum GKB7 (NITE BP-03889) as an effective ingredient.

In the foregoing embodiments, the subject may be an adult, and the effective dose of the complex probiotics administered to the subject can be 50 mg/60 kg body weight/day to 300 mg/60 kg body weight/day.

In the foregoing embodiments, the subject can be a mouse, and the effective dose of the complex probiotics administered to the subject can be 10.5 mg/kg/day to 63 mg/kg/day.

Application of the aforementioned complex probiotic composition and a method for improving the exercise performance of the subject with the low intrinsic aerobic exercise capacity, which includes Lactobacillus rhamnosus GKLC1, Bifidobacterium lactis GKK24 and Clostridium butyricum GKB7, administered to the subject with the low intrinsic aerobic exercise capacity in the continuation period, can effectively reduce serum lactic acid and serum urea nitrogen after aerobic exercise, reduce proportion of offal fat and/or increase liver and muscle glycogen contents, thereby being as an effective ingredient for preparation of various compositions.

It can be understood that the foregoing general description and the following detailed description are only examples, and are intended to provide further explanations to the claimed invention.

### BRIEF DESCRIPTION OF THE DRAWINGS

In order to allow the above-mentioned and other objects, features, advantages and embodiments of the present disclosure to be more clearly understood, the detailed description of the accompanying drawings is as follows:

FIG. 1 is a bar graph showing forelimb grip strength of mice in a control group and experimental groups according to an embodiment of the present disclosure.

FIG. 2 is a bar graph showing weight-bearing swimming times to exhaustion of mice in a control group and experimental groups according to an embodiment of the present disclosure.

FIG. 3 is a bar graph showing an oral glucose tolerance test of mice in a control group and experimental groups according to an embodiment of the present disclosure.

FIG. 4 is a bar graph showing levels of serum lactic acid of mice after exercise in a control group and experimental groups according to an embodiment of the present disclosure.

FIG. 5 is a bar graph showing levels of serum lactic acid of mice after exercise and after rest in a control group and experimental groups according to an embodiment of the present disclosure.

FIG. 6 is a bar graph showing levels of serum urea nitrogen of mice after exercise and after rest in a control group and experimental groups according to an embodiment of the present disclosure.

FIG. 7 is a bar graph showing liver glycogen contents of mice in a control group and experimental groups according to an embodiment of the present disclosure.

FIG. 8 is a bar graph showing muscle glycogen contents of mice in a control group and experimental groups according to an embodiment of the present disclosure.

FIG. 9 is a bar graph showing weights of epididymal fat in mice in a control group and experimental groups according to an embodiment of the present disclosure.

### DETAILED DESCRIPTION

If the definition or usage of a term in the cited document is inconsistent with or contrary to the definition of the term here, the definition of the term here applies instead of the definition of the term in the cited document. Furthermore, unless otherwise defined by context, the singular term can include plural, and the plural term can include singular.

As mentioned above, the present disclosure provides a complex probiotic composition and a method for improving exercise performance of a subject with low intrinsic aerobic exercise capacity. After being administered to the subject in a continuation period, it can effectively reduce serum lactic acid and serum urea nitrogen after aerobic exercise, increase liver glycogen content and muscle glycogen content, and/or reduce proportion of offal fat.

In one embodiment, “complex probiotics” referred to herein can refer to probiotics including different species or genus, for example, including Lactobacillus rhamnosus GKLC1, Bifidobacterium lactis GKK24, and Clostridium butyricum GKB7, and the Lactobacillus rhamnosus GKLC1 is deposited in the China General Microbiological Culture Collection Center (CGMCC, No. 3, Yard 1, West Beichen Road, Chaoyang District, Beijing, China) on Jan. 12, 2018 and assigned accession number CGMCC 15202, and the Bifidobacterium lactis GKK24 is deposited in the International Patent Organism Depositary, National Institute of Technology and Evaluation (NITE-IPOD, #122, 2-5-8 Kazusakamatari, Kisarazu-shi, Chiba, Japan) on Oct. 14, 2022 and assigned accession number NITE BP-03766, and the Clostridium butyricum GKB7 is deposited in the NITE-IPOD on May 22, 2023 and assigned accession number NITE BP-03889. It should be added that the present disclosure requires selection of the specific strains of complex probiotics, so that the obtained complex probiotic composition can exert effects such as improving the exercise performance of the subject with the low intrinsic aerobic exercise capacity. If the complex probiotics are not selected from the above-mentioned specific strains, and the strains are increased or decreased, or some or all of them are replaced with other strains of the same species, it cannot be expected to improve the exercise performance of the subject with the low intrinsic aerobic exercise capacity (AEC).

The “subject with low intrinsic aerobic exercise capacity” referred to here is based on a method published by Koch and Britton (2001; Physiol. Genomics 5(1): 45-52) that swimming endurance time of mice is used as a screening index for high intrinsic (H) or low intrinsic (L) aerobic exercise capacity (AEC). In one embodiment, for a single mating, male and female individuals ranked in the bottom 20% of the aerobic exercise capacity of the group are selected for breeding of a next generation. Breeding could be carried out at least once, but a population that had been bred more than 5 times (inclusive) is more stable, and a population that had been bred more than 8 times (inclusive) is more stable. In a specific example, a population of the 8th generation of mating could be regarded as subjects with low intrinsic aerobic exercise capacity (L-AEC) for subsequent evaluation of exercise performance.

The evaluation item of “exercise performance” referred to herein can include, but not limited to a level of serum lactic acid and a level of serum urea nitrogen after aerobic exercise, proportion of offal fat and/or liver and muscle glycogen contents. Generally, the exercise performance of the L-AEC subjects can include, but not limited to increasing the level of serum lactic acid and the level of serum urea nitrogen after aerobic exercise, increasing offal fat and/or reducing liver and muscle glycogen contents. Therefore, in one embodiment, an oral composition containing an effective dose of complex probiotics administered to the L-AEC subjects for at least 28 consecutive days can significantly improve the exercise performance of the L-AEC subjects, including but not limited to reducing the serum lactic acid after aerobic exercise, reducing the serum urea nitrogen after aerobic exercise, increasing the liver glycogen content, increasing the muscle glycogen content and/or reducing the proportion of the offal fat. In other embodiments, the exercise performance of the L-AEC subjects improved by the above-mentioned complex probiotics can optionally include improving forelimb grip strength, improving aerobic exercise performance, prolonging aerobic exercise time to exhaustion (or called prolonging weight-bearing swimming time to exhaustion), increasing ability to metabolize glucose (or called increasing glucose tolerance), etc.

In one embodiment, there is no specific limitation to a content of each strain of the above-mentioned complex probiotics. In one example, a weight (mg) ratio or a CFU ratio of the Lactobacillus rhamnosus GKLC1 (CGMCC 15202), the Bifidobacterium lactis GKK24 (NITE BP-03766) and the Clostridium butyricum GKB7 (NITE BP-03889) can be, for example, 1:1:1; however, in other examples, the weight (mg) ratio or the CFU ratio of the Lactobacillus rhamnosus GKLC1 (CGMCC 15202), the Bifidobacterium lactis GKK24 (NITE BP-03766) and the Clostridium butyricum GKB7 (NITE BP-03889) can be other than 1:1:1, such as 1:1:(>1˜4).

In practice, the composite probiotic composition can be, for example, an oral composition, such as a pharmaceutical composition or a food composition. A usage of the complex probiotics can include, but not limited to, whole fermented liquid, bacterium mud (or called cell pellet), supernatant, and lyophilized powder. In the above-mentioned embodiments, the aforementioned whole fermented liquid refers to a product including bacteria and culture solution. The aforementioned bacterium mud refers to a product after removing the supernatant from the whole fermentation liquid. The aforementioned supernatant refers to a product after removing the bacterium mud from the whole fermented liquid. The aforementioned lyophilized powder refers to the lyophilized powder prepared from the whole fermented liquid, the bacterium mud and/or the supernatant.

Examples of the aforementioned pharmaceutical composition can include, but be not limited to, pharmaceuticals. Examples of the complex probiotics applied to the food composition can include, but be not limited to, general foods, health foods, beverages, nutritional supplements, dairy or feed, etc. In examples of the aforementioned oral composition, the composite probiotic composition can further optionally include a pharmaceutical and food-acceptable carrier, excipient and/or additive. In other examples, a dosage form of the complex probiotic composition can include, but not limited to, powder, lozenge, granule, suppository, microcapsule, ampoule, liquid spray or suppository.

When the complex probiotics are used to prepare a composition for improving the exercise performance of the subject with the low intrinsic aerobic exercise capacity, the oral composition containing the effective dose of the complex probiotics can be administered to the subject for a continuous period of time. The effective dose depends on the subject and is not particularly limited. For example, when the subject is an adult, the effective dose of the complex probiotics for the adult can be, for example, 50 mg/60 kg body weight/day to 300 mg/60 kg body weight/day, and preferably 75 mg/60 kg body weight/day to 200 mg/60 kg body weight/day, and more preferably about 100 mg/60 kg body weight/day. In another example, when the subject is a mouse, the effective dose of the complex probiotics for the subject can be, for example, 10.5 mg/kg/day to 63 mg/kg/day, and preferably 15.75 mg/kg/day to 42 mg/kg/day, and more preferably about 21 mg/kg/day. In other examples, the administration period can be, for example, at least 28 consecutive days, or longer or shorter duration.

It should be understood that following specific bacterial strains, specific formulations, specific dosages, specific detection methods, viewpoints, illustrations and examples are for illustrative purposes only, and are not intended to be limitations of the present disclosure. Principal features of the present disclosure can be applied in various embodiments without departing from the spirit and scope of the present disclosure. Therefore, those skilled in the art of the present disclosure can easily determine essential technical features of the present disclosure, and make various changes and modifications to the present disclosure to apply to different purposes and conditions without departing from the spirit and scope of the present disclosure.

### Example 1

### 1.1 Sources of Bacteria

The strains used in the example included a specific combination of probiotics, including Lactobacillus rhamnosus GKLC1, Bifidobacterium lactis GKK24 and Clostridium butyricum GKB7. The Lactobacillus rhamnosus GKLC1 was deposited in the China General Microbiological Culture Collection Center (CGMCC, No. 3, Yard 1, West Beichen Road, Chaoyang District, Beijing, China) on Jan. 12, 2018 and assigned accession number CGMCC 15202. The Bifidobacterium lactis GKK24 was deposited in the International Patent Organism Depositary, National Institute of Technology and Evaluation (NITE-IPOD, #122, 2-5-8 Kazusakamatari, Kisarazu-shi, Chiba, Japan) on Oct. 14, 2022 and assigned accession number NITE BP-03766. The Clostridium butyricum GKB7 was deposited in NITE-IPOD on May 22, 2023 and assigned accession number NITE BP-03889.

The strain GKLC1, strain GKK24 and strain GKB7 were all isolated from human body. For example, the strain GKLC1 was isolated from mother's milk. The strain GKK24 was isolated from infant feces, and the strain GKB7 was isolated from feces. After DNA of the strain GKK24 and that of the strain GKB7 were purified, gene sequencing was carried out to obtain nucleic acid fragment sequences of 16s rRNA gene of the strain GKK24 and that of the strain GKB7, as shown in SEQ ID NO:1 and SEQ ID NO:2, respectively. After sequence alignment, the strain GKK24 was identified as the Bifidobacterium lactis, and the strain GKB7 was identified as the Clostridium butyricum. The above-mentioned DNA purification, gene sequencing and sequence alignment were carried out using conventional methods, which did not affect subsequent evaluations of improving intrinsic aerobic exercise capacity by the strain GKK24, so details were not repeated here. The Bifidobacterium lactis GKK24 was Gram-positive, acid-fast-staining negative, obligately anaerobic, non-motile, without spores, without capsule and flagella. The Clostridium butyricum GKB7 was Gram-positive, obligately anaerobic, motile, with spores and flagella.

In some examples, the Lactobacillus rhamnosus GKLC1, the Bifidobacterium lactis GKK24 and the Clostridium butyricum GKB7 were respectively inoculated on solid media to activate the strains. In a preferred example, the solid medium was commercially available MRS (deMan, Rogosa and Sharpe) agar or RCM (reinforced clostridial medium) agar. After colonies were generated, a single colony was picked out and inoculated into liquid medium for liquid culture. In a preferred specific example, the strain was cultivated at a temperature of 25° C. to 40° C. In a preferred embodiment, time for liquid culture was 16 to 24 hours. In a preferred example, the liquid medium was MRS liquid medium or RCM liquid medium. After the liquid culture was completed, fermentation culture was carried out. In a preferred specific example, a formulation of the fermentation medium was listed in Table 1 below.

TABLE 1
Formulation of Fermentation Medium

| Component | Content (wt. %) |
|---|---|
| sugar | 1~10 |
| yeast extract | 0.1~5 |
| protein peptone | 0.1~5 |
| trace elements | 0.01~2 |
| cysteine | 0.01~0.1 |
| Tween-80 | 0.05~1 |

### 1.2 Preparation of Lyophilized Powder

After the strain GKLC1, the strain GKK24 and the strain GKB7 had been fermented and grown, respectively, whole fermented liquid containing bacteria and culture solution was collected and centrifuged with a speed of 1,000 rpm to 15,000 rpm to obtain bacterium mud. The obtained bacterium mud was mixed with a protective agent (i.e., 6 wt. % to 50 wt. % of skimmed milk powder) and then lyophilized. The lyophilization was performed by pre-freezing the mixture in a gradient setting mode that was stored at 20° C. to 0° C. for 1 to 4 hours, then stored at 0° C. to −20° C. for 4 to 8 hours, and then stored at −196° C. to −30° C. for more than 8 hours. In a preferred example, the lyophilization was performed by storing the mixture at −40° C. for 2 hours, then storing it at −20° C. for 2 hours, then storing it at 0° C. for 2 hours, and finally storing it at 20° C. for about 10 hours or more. Afterwards, the obtained lyophilized powder was stored at low temperature. In another preferred example, the low storage temperature was −30° C. to 4° C. The preserved lyophilized powder could be used as a raw material of the complex probiotic composition to be administered to experimental animals in following animal experiments.

### 1.3 Experimental Animals

A total of twenty-four 8-week-old ICR male mice with low intrinsic aerobic exercise capacity were selected and used in following examples, each weighing about 35 grams, purchased from BioLASCO Co., Ltd. The mice were kept in standard breeding cages with 3 mice per cage, and average temperature of the mouse room was about 22±2° C., and average humidity was 65±5%, and light and darkness were regularly 12 hours, and drinking water and standard feed were adequately supplied. All animal experimental procedures were reviewed and approved by Institutional Animal Care and Use Committee (IACUC) of National Taiwan Sport University in Taiwan, China, and approval number IACUC-11011 was obtained. The experimental animals were grouped according to their body weights arranged in S shape, and then divided into one control group and three experimental groups, with six animals in each group.

### 1.4 Breeding of Mice with Low Intrinsic Aerobic Exercise Capacity

According to the method published by Koch and Brittion (2001), swimming endurance time of mice was screened by an index for high intrinsic (H) or low intrinsic (L) aerobic exercise capacity (AEC). For a single mating, male and female individuals ranked in the bottom 20% of the aerobic exercise capacity of the group were selected for breeding of a next generation. A total of eight breedings were done, and a population of the 8th generation was considered as subjects with low intrinsic aerobic exercise capacity (L-AEC).

### 1.5 Screening Method for Mice with Low Intrinsic Aerobic Exercise Capacity

An exhaustive swimming endurance test was carried out after the mice were adapted for one week. In the test, a lead sheet with 5% of the body weight of the mouse was fixed to base of a tail of the mouse, and the mouse was placed in a bucket with a water depth of 40 cm and a surface diameter of 45 cm, and water temperature was controlled at 28±1° C., to let the mouse continue to swim to exhaustion, and the mouse was immediately rescued to avoid drowning. In the test, swimming exhaustion was defined as the mouse sinking upright or being unable to swim back to a water surface for breathing within 7 seconds after sinking into water. The exhaustive swimming endurance test was carried out twice with an interval of 72 hours between each time to allow the mice to recover their physical strength. The better result of the two swimming results of the individual was took as a comparison with others. The performance of the aerobic endurance exercise was expressed by swimming time to exhaustion of the mice; the worse the performance of the aerobic endurance exercise, the shorter the swimming time to exhaustion.

### 1.6 Experimental Design

The experimental animals were orally administered with sterilized water (the control group) or the probiotic composition of Example 1 (the experimental groups) for four consecutive weeks (i.e., 28 days). The effective dose of the complex probiotics administered to the mice of the experimental groups was 21 mg/kg/day (equivalent to an adult with an effective dose of 100 mg/60 kg body weight/day). After four weeks of ingestion, following analyzes were performed: forelimb grip strength (unit: g), weight-bearing swimming time to exhaustion (unit: minute), a level of blood glucose (mg/dL) in an oral glucose tolerance test, a level of serum lactic acid (unit: mmol/L) after exercise, a level of serum lactic acid (unit: mmol/L) after exercise and after rest, a level of serum urea nitrogen (unit: mg/dL) after exercise and after rest, liver and muscle glycogen contents (mg/g liver or mg/g muscle) and a weight of epididymal fat (which could represent proportion of offal fat tissue).

### 1.7 Statistics

Numerical values described herein were represented by mean±standard deviation (mean±SD), and analyzed using one-way ANOVA and commercially available software, with p<0.05 to determine that there was statistical significance difference, in which the symbol “**” represented p<0.05, and the symbol “**” represented p<0.005.

### Example 2. Evaluation of Complex Probiotics of Example 1 for Improving Exercise Performance of L-AEC Subjects

### 2.1 Evaluation of Forelimb Grip Strength

In the example, a forelimb grip test was carried out 30 minutes after feeding the probiotic composition containing Example 1 on the 29th day. At first, the base of the tail of the mouse was grasped and lowered vertically, so that two forelimbs grasped a crossbar (2 mm in diameter and 7.5 cm in length) connecting to AIKOH electronic push-pull force gauge (Model-RX-5, Aikoh Engineering, Nagoya, Japan), and the tail of the mouse was slightly pulled back to make the forelimbs of the mouse leave the cross bar, and a maximum value of the forelimb grip strength of the mouse recorded by the push-pull force gauge during the experimentation could be as an index for evaluating the grip strength.

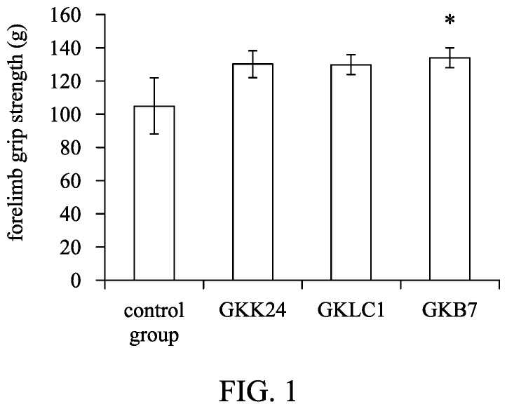

Please refer to FIG. 1, which was a bar graph showing the forelimb grip strength (g) of the mice in the control group and the experimental groups according to an embodiment of the present disclosure.

As shown in FIG. 1, the forelimb grip strength of the mice in the control group was about 105 g, and the forelimb grip strength of the mice in the oral administration group of GKK24 was about 130 g, and the forelimb grip strength of the mice in the oral administration group of GKLC1 was about 130 g, and the forelimb grip strength of the mice in the oral administration group of GKB7 was about 134 g, which proved that the forelimb grip strength of the L-AEC mice after the probiotic strains of the present disclosure were administered to the L-AEC mice for four consecutive weeks were indeed significantly improved.

### 2.2 Evaluation of Exercise Endurance—Weight-bearing Swimming Time to Exhaustion

After the forelimb grip test was completed, a lead sheet with 5% of the body weight of the mouse was fixed to the base of the tail of the mouse, and the mouse was subjected to a weight-bearing swimming exhaustion test. The animals were allowed to adapt to swimming one week before the weight-bearing swimming exhaustion test, and they were in an environment with a diameter of 28 cm, a water depth of 25 cm, and water temperature of 27±1° C. The test was carried out in a single swimming mode, and the mouse was put into a water tank, and the test animal was forced to swim. The limbs of the mouse were kept in motion throughout the experiment. If the mouse was floating on the water surface and the limbs were not moving, a stir bar was used to stir water near the mouse. The performance of the exercise endurance (weight-bearing swimming to exhaustion) was shown by recording the time until the head of the mouse was completely submerged in water for 8 seconds and did not surface.

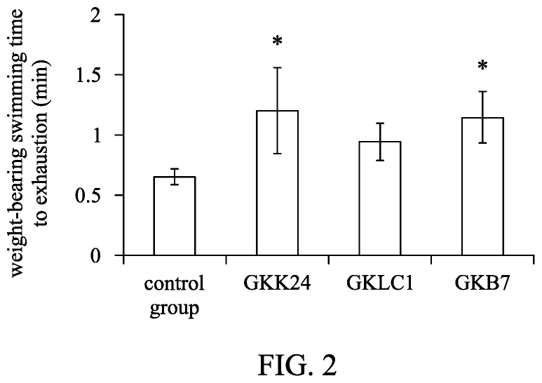

Please refer to FIG. 2, which was a bar graph showing the weight-bearing swimming time to exhaustion (minutes) of the mice in the control group and the experimental groups according to an embodiment of the present disclosure.

As shown in FIG. 2, the weight-bearing swimming times to exhaustion of the mice of the control group were about 0.65 minutes, and the weight-bearing swimming times to exhaustion of the mice of the oral administration group of GKK24 were about 1.20 minutes, and the weight-bearing swimming times to exhaustion of the mice of the oral administration group of GKLC1 were about 0.94 minutes, and the weight-bearing swimming times to exhaustion of the mice of the oral administration group of GKB7 were about 1.15 minutes, which proved that the exercise endurance of the L-AEC mice after the probiotic strains of the present disclosure were administered to the L-AEC mice for four consecutive weeks could be significantly improved.

### 2.3 Oral Glucose Tolerance Test (OGTT)

An oral glucose tolerance test was used to evaluate glucose metabolism ability of the mice with lineage of the low aerobic exercise after the strains of the present disclosure were administered for four weeks. After 25% glucose solution was prepared, 2.5 g/kg body weight (BW) was administered to the mouse that had fasted for 12 hours, and blood glucose values of the mouse at time points 0, 15, 30, 60, and 120 minutes were analyzed using Roche blood glucose meter (AccuChek®, Germany).

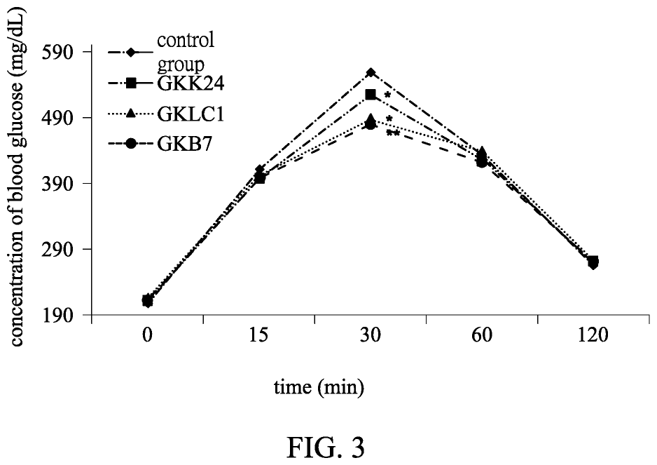

Please refer to FIG. 3, which was a bar graph showing the oral glucose tolerance test of the mice in the control group and the experimental groups according to an embodiment of the present disclosure.

As shown in FIG. 3, after oral administration of glucose, changes in blood glucose (mg/dL, the vertical axis) of the mice administered with the strain GKK24, the strain GKLC1, the strain GKB7 were lower than those of the mice of the control group, which proved that the probiotic strains of the present disclosure administered to the L-AEC mice for four consecutive weeks indeed beneficially improved the glucose tolerance of the L-AEC mice.

### 2.4 Evaluation of Level of Serum Lactic Acid After Exercise and After Rest

After the mice in each group were respectively swimming without weight-bearing for 10 minutes and having a rest for 20 minutes, blood samples were collected and then centrifuged at 1,500×g at 4° C. for 10 minutes, and a fully automatic biochemical analyzer (Hitachi 7060, Hitachi, Tokyo, Japan) was used to analyze levels of serum lactic acid.

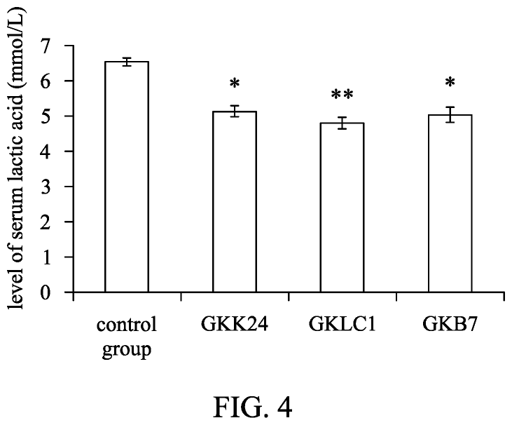

Please refer to FIG. 4, which was a bar graph showing the levels of serum lactic acid (mmol/L) of the mice after exercise in the control group and the experimental groups according to an embodiment of the present disclosure.

FIG. 4 shows the levels of serum lactic acid of the mice after exercise. The levels of serum lactic acid of the mice in the control group were about 6.55 mmol/L, and the levels of serum lactic acid of the mice in the oral administration group of GKK24 were about 5.15 mmol/L, and the levels of serum lactic acid of the mice in the oral administration group of GKLC1 were about 4.81 mmol/L, and the levels of serum lactic acid of the mice in the oral administration group of GKB7 were about 5.04 mmol/L, which proved that accumulation of serum lactic acid after exercise could be significantly reduced after the probiotic strains of the present disclosure were administered to the L-AEC mice for four consecutive weeks.

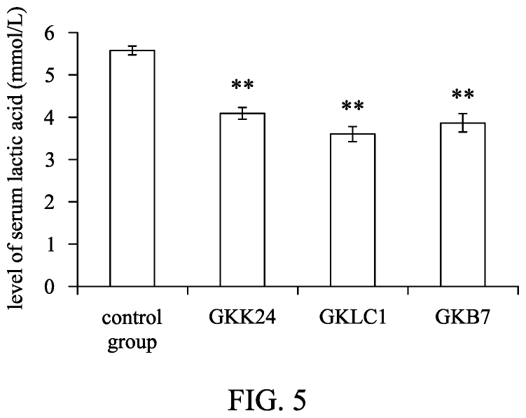

Please refer to FIG. 5, which was a bar graph showing the levels of serum lactic acid (mmol/L) of the mice after exercise and after rest in the control group and the experimental groups according to an embodiment of the present disclosure.

As shown in FIG. 5, after the mice exercised and rested, the levels of serum lactic acid of the mice in the control group were about 5.59 mmol/L, and the levels of serum lactic acid of the mice in the oral administration group of GKK24 were about 4.10 mmol/L, and the levels of serum lactic acid of the mice in the oral administration group of GKLC1 were about 3.60 mmol/L, and the levels of serum lactic acid of the mice of the oral administration group of GKB7 were about 3.87 mmol/L, which proved that a clearance effect of serum lactic acid at rest after exercise could be significantly improved after the probiotic strains of the present disclosure were administered to the L-AEC mice for four consecutive weeks.

### 2.5 Evaluation of Level of Serum Urea Nitrogen After Exercise and After Rest

After the mice in each group were respectively swimming without weight-bearing for 90 minutes and having a rest for 60 minutes, blood samples were collected and then centrifuged at 1500×g, 4° C. for 10 minutes, and the fully automatic biochemical analyzer (Hitachi 7060, Hitachi, Tokyo, Japan) was used to analyze levels of serum urea nitrogen.

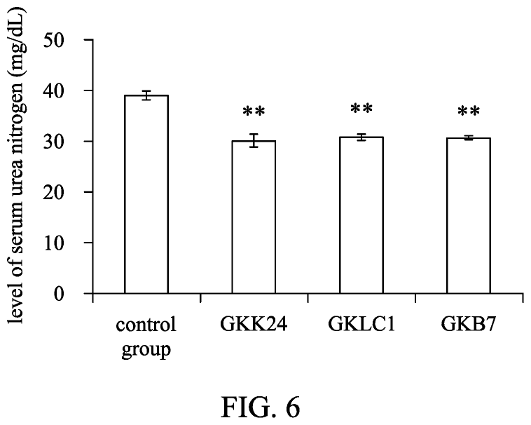

Please refer to FIG. 6, which was a bar graph showing the levels of serum urea nitrogen (mg/dL) of the mice after exercise and after rest in the control group and the experimental groups according to an embodiment of the present disclosure.

As shown in FIG. 6, the levels of serum urea nitrogen of the mice in the control group were about 39.10 mg/dL, and the levels of serum urea nitrogen of the mice in the oral administration group of GKK24 were about 30.15 mg/dL, and the levels of serum urea nitrogen of the mice in the oral administration group of GKLC1 were about 30.85 mg/dL, and the levels of serum urea nitrogen of the mice in the oral administration group of GKB7 were about 30.70 mg/dL, which proved that the level of serum urea nitrogen at rest after exercise could be significantly reduced after the probiotic strains of the present disclosure were administered to the L-AEC mice for four consecutive weeks.

### 2.6 Evaluation of Glycogen Contents in Liver and Muscle Tissues

After the tests, liver and muscle tissues of the mice were taken to analyze glycogen contents. 100 mg of the liver or muscle tissue was centrifuged with 0.5 mL of 10% glacial perchloric acid at 15,000×g at 4° C. for 15 minutes, and 30 μL of supernatant was taken and added to a 96-well plate, and mixed with 200 μL of iodine-potassium iodide reagent to make iodine combine with glycogen for 10 minutes to turn brown, and ELISA (Tecan Infinite M200, Tecan Austria, Salzburg, Austria) was used to detect absorption wavelength at 460 nm. Standard glycogen purchased from Sigma was used as a standard curve to convert and detect the glycogen contents (mg/g tissue) in the tissues.

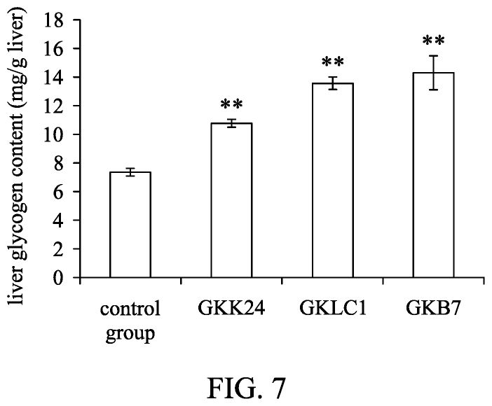

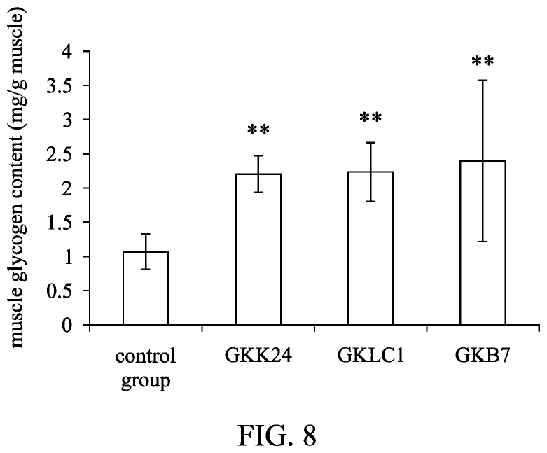

Please refer to FIG. 7 and FIG. 8, which were bar graphs respectively show the glycogen contents (mg/g liver) in the liver (FIG. 7) and in the muscle (FIG. 8) of the mice in the control group and the experimental groups according to an embodiment of the present disclosure.

As shown in FIG. 7, the glycogen contents in the livers of the mice in the control group were about 7.36 mg/g liver, and the glycogen contents in the livers of the mice in the oral administration group of GKK24 were about 10.77 mg/g liver, and the glycogen contents in the livers of the mice in the oral administration group of GKLC1 were about 13.57 mg/g liver, and the glycogen contents in the livers of the mice in the oral administration group of GKB7 were about 14.31 mg/g liver, which proved that the glycogen content in the liver could be significantly increased after the probiotic strains of the present disclosure were administered to the L-AEC mice for four consecutive weeks.

As shown in FIG. 8, the glycogen contents in the muscles of the mice in the control group were about 1.07 mg/g muscle, and the glycogen contents in the muscles of the mice in the oral administration group of GKK24 were about 2.21 mg/g muscle, and the glycogen contents in the muscles of the mice in the oral administration group of GKLC1 were about 2.24 mg/g muscle, and the glycogen contents in the muscles of the mice in the oral administration group of GKB7 were about 2.24 mg/g muscle, which proved that the glycogen content in the muscle could be significantly increased after the probiotic strains of the present disclosure were administered to the L-AEC mice for four consecutive weeks.

### 2.7 Evaluation of Weight of Epididymal Fat

After the tests, epididymides of the mice were taken and weighed, weights of epididymal fat tissues were recorded, which could represent proportion of offal fat tissue.

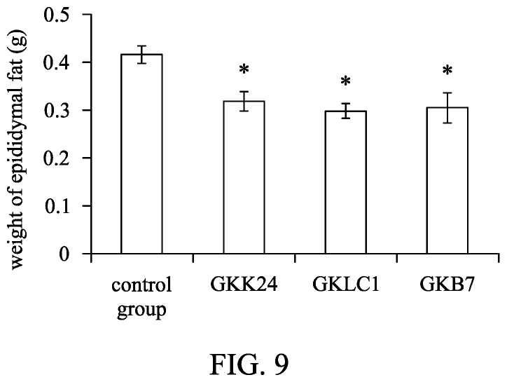

Please refer to FIG. 9, which was a bar graph showing the weights (g) of the epididymal fat of the mice in the control group and the experimental groups according to an embodiment of the present disclosure.

As shown in FIG. 9, the weights of epididymal fat of the mice in control group were about 0.42 g, and the weights of epididymal fat of the mice in the oral administration group of GKK24 were about 0.32 g, and the weights of epididymal fat of the mice in the oral administration group of GKLC1 were about 0.30 g, and the weights of epididymal fat of the mice in the oral administration group of GKB7 were about 0.31 g, which proved that the proportion of the offal fat tissue could be significantly reduced after the probiotic strains of the present disclosure were administered to the L-AEC mice for four consecutive weeks.

In summary, the specific bacterial strains, specific formulations, specific dosages, specific detection methods, or specific evaluation methods of the present disclosure are only used to illustrate the complex probiotic composition and the method for improving the exercise performance of the subject with the low intrinsic aerobic exercise capacity. However, those skilled in the art of the present disclosure should understand that without departing from the spirit and scope of the present disclosure, two or three of the Lactobacillus rhamnosus GKLC1, the Bifidobacterium lactis GKK24 and the Clostridium butyricum GKB7, other formulations, other dosages, other detection methods, or other evaluation methods can also be used to improve the complex probiotic composition and the method for improving the exercise performance of the subject with the low intrinsic aerobic exercise capacity, and are not limited to above. For example, the complex probiotic composition can be the pharmaceutical composition or the food composition, which can optionally include the pharmaceutical and food-acceptable carrier, excipient and/or additive, and can be made into the dosage form of the powder, tablet, granule, suppository, microcapsule, ampoule, liquid spray or suppository.

According to the foregoing embodiments, the complex probiotic composition for improving the exercise performance of the subject with the low intrinsic aerobic exercise capacity of the present disclosure has the advantages that the complex probiotic composition including the Lactobacillus rhamnosus GKLC1, the Bifidobacterium lactis GKK24 and the Clostridium butyricum GKB7, administered to the subject with the low intrinsic aerobic exercise capacity in a continuation period, can effectively reduce serum lactic acid and serum urea nitrogen after aerobic exercise, reduce proportion of offal fat and/or increase liver and muscle glycogen contents, thereby being as the effective ingredient for preparation of various compositions.

While the present disclosure has been disclosed above with several specific embodiments, other embodiments are possible. Therefore, the spirit and scope of the appended claims of the present disclosure should not be limited to the description of the embodiments contained herein.

What is claimed is:

1. A complex probiotic composition for improving exercise performance of a subject with low intrinsic aerobic exercise capacity, characterized in that it comprises Lactobacillus rhamnosus GKLC1, Bifidobacterium lactis GKK24 and Clostridium butyricum GKB7, and the Lactobacillus rhamnosus GKLC1 was deposited in the China General Microbiological Culture Collection Center on Jan. 12, 2018 and assigned accession number CGMCC 15202, and the Bifidobacterium lactis GKK24 was deposited in the International Patent Organism Depositary, National Institute of Technology and Evaluation, Japan on Oct. 14, 2022 and assigned accession number NITE BP-03766, and the Clostridium butyricum GKB7 was deposited in the International Patent Organism Depositary, National Institute of Technology and Evaluation, Japan on May 22, 2023 and assigned accession number NITE BP-03889.

2. The complex probiotic composition for improving the exercise performance of the subject with the low intrinsic aerobic exercise capacity of claim 1, wherein the complex probiotic composition is an oral composition.

3. The complex probiotic composition for improving the exercise performance of the subject with the low intrinsic aerobic exercise capacity of claim 2, wherein the complex probiotic composition is a pharmaceutical composition or a food composition.

4. The complex probiotic composition for improving the exercise performance of the subject with the low intrinsic aerobic exercise capacity of claim 2, further comprising a pharmaceutical and food-acceptable carrier, excipient and/or additive.

5. The complex probiotic composition for improving the exercise performance of the subject with the low intrinsic aerobic exercise capacity of claim 1, wherein a dosage form of the complex probiotic composition comprises powder, lozenge, granule, suppository, microcapsule, ampoule, liquid spray, or suppository.

6. The complex probiotic composition for improving the exercise performance of the subject with the low intrinsic aerobic exercise capacity of claim 1, wherein improving the exercise performance of the subject with the low intrinsic aerobic exercise capacity comprises reducing a level of serum lactic acid after aerobic exercise.

7. The complex probiotic composition for improving the exercise performance of the subject with the low intrinsic aerobic exercise capacity of claim 1, wherein improving the exercise performance of the subject with the low intrinsic aerobic exercise capacity comprises reducing a level of serum urea nitrogen after aerobic exercise.

8. The complex probiotic composition for improving the exercise performance of the subject with the low intrinsic aerobic exercise capacity of claim 1, wherein improving the exercise performance of the subject with the low intrinsic aerobic exercise capacity comprises reducing proportion of offal fat.

9. The complex probiotic composition for improving the exercise performance of the subject with the low intrinsic aerobic exercise capacity of claim 1, wherein improving the exercise performance of the subject with the low intrinsic aerobic exercise capacity comprises increasing liver glycogen content.

10. The complex probiotic composition for improving the exercise performance of the subject with the low intrinsic aerobic exercise capacity of claim 1, wherein improving the exercise performance of the subject with the low intrinsic aerobic exercise capacity comprises increasing muscle glycogen content.

11. A method of improving exercise performance of a subject with low intrinsic aerobic exercise capacity, comprising administering an oral composition containing an effective dose of the complex probiotics to a subject with low intrinsic aerobic exercise capacity for at least 28 consecutive days, and the complex probiotics comprise Lactobacillus rhamnosus GKLC1 (CGMCC 15202), Bifidobacterium lactis GKK24 (NITE BP-03766) and Clostridium butyricum GKB7 (NITE BP-03889) as an effective ingredient.

12. The method of claim 11, wherein improving the exercise performance of the subject with the low intrinsic aerobic exercise capacity comprises reducing serum lactic acid after aerobic exercise.

13. The method of claim 11, wherein improving the exercise performance of the subject with the low intrinsic aerobic exercise capacity comprises reducing serum urea nitrogen after aerobic exercise.

14. The method of claim 11, wherein improving the exercise performance of the subject with the low intrinsic aerobic exercise capacity comprises reducing proportion of offal fat.

15. The method of claim 11, wherein improving the exercise performance of the subject with the low intrinsic aerobic exercise capacity comprises increasing liver glycogen content.

16. The method of claim 11, wherein improving the exercise performance of the subject with the low intrinsic aerobic exercise capacity comprises increasing muscle glycogen content.

17. The method of claim 11, wherein the subject is an adult, and the effective dose of the complex probiotics administered to the subject is 50 mg/60 kg body weight/day to 300 mg/60 kg body weight/day.

18. The method of claim 11, wherein the subject is a mouse, and the effective dose of the complex probiotics administered to the subject is 10.5 mg/kg/day to 63 mg/kg/day.


SEQUENCE LISTING (2 sequences)

SEQ ID NO: 1 - DNA, 1050 bp, Bifidobacterium lactis GKK24
  source 1..1050: mol_type = other DNA
gggtggtctcgtccgcttcctgcagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattggggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctgggatgtggggccctttccacgggtcccgtgtcggagccaacgcgttaagcatcccgcctggggagtacgggccgcaaggctaaaactcaaagaaattgacgggggcccgcacaagcggcggagcatgcggattaattcgatgcaacgcgaagaccttacctgggcttgacatgtgccgggatcgcgtgagacacgttttcccttcggggcgatcacaggtgtgcatgtcgtcgtcagctcgtgtcgtgagatgtgggtagtcccg

SEQ ID NO: 2 - DNA, 1288 bp, Clostridium butyricum GKB7
  source 1..1288: mol_type = other DNA
ggggaagtggcgggcgtgcttacacatgcagtcgagcgatgaagctccttcgggagtggattagcggcggacgggtgagtaacacgtgggtaacctgcctcatagaggggaatagcctttcgaaaggaagattaataccgcataagattgtagtaccgcatggtacagcaattaaaggagtaatccgctatgagatggacccgcgtcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggtcttcggattgtaaagctctgtctttagggacgataatgacggtacctaaggaggaagccacggctaactacgtgccagcagccgcggtaatacgtaggtggcaagcgttgtccggatttactgggcgtaaagggagcgtaggtggatatttaagtgggatgtgaaatacccgggcttaacctgggtgctgcattccaaactggatatctagagtgcaggagaggaaaggagaattcctagtgtagcggtgaaatgcgtagagattaggaagaataccagtggcgaaggcgcctttctggactgtaactgacactgaggctcgaaagcgtggggagcaaacaggattagataccctggtagtccacgccgtaaacgatgaatactaggtgtaggggttgtcatgacctctgtgccgccgctaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgaattactctgtaatgaggagccacttcgtgcagagacaggtgtgcatggtgtcgtcagctcgtgtcgtgagatgttgggttaagtcccgcacgagcgcacccttatgtagtgctaccatttagtgagcactctagcgagactgccgggttaccgggagaaagggggggatgacgtccaatcatcatgccccttattgtctaggctaccacacgtgcttacaatgggtcggtacaatgaaaatgcaccctcgcgaagtgaacaaccataaaccgacttcaagctcggattgtagggtggaactcgcgcctctacataaaccgtggaaggtggtcttaataaatc